(19) United States
(12) Patent Application Publication
(10) Pub. No.: US 2009/0118620 A1
(43) Pub. Date: May 7, 2009

(54) SYSTEM AND METHOD FOR TRACKING AN ULTRASOUND CATHETER

(75) Inventors: Nora T. Tgavalekos, Tewksbury, MA (US); Peter Traneus Anderson, Andover, MA (US); Dun Alex Li, Salem, NH (US); Jonathan David Schiff, Andover, MA (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 12/200,587

(22) Filed: Aug. 28, 2008

Related U.S. Application Data
(63) Continuation-in-part of application No. 11/935,479, filed on Nov. 6, 2007.

(51) Int. Cl. A61B 8/14

(52) U.S. Cl. 600/463

(57) ABSTRACT

A surgical navigation system including an ultrasound catheter is disclosed herein. The ultrasound catheter includes a flexible catheter housing defining a distal end, a transducer array disposed at least partially within the catheter housing, and a motor coupled with the transducer array. The motor is configured to rotate the transducer array in order to image a three-dimensional (3D) volume. The ultrasound catheter also includes at least one tracking element adapted to provide an estimate of a position and orientation of the distal end of the catheter housing. The at least one tracking element is disposed within the catheter housing and located immediately adjacent to the distal end.

### CROSS-REFERENCE TO RELATED APPLICATIONS

This application is a continuation-in-part of and claims priority to U.S. patent application Ser. No. 11/935,479, filed on Nov. 6, 2007, the disclosure of which is incorporated herein by reference.

### BACKGROUND OF THE INVENTION

This disclosure relates generally to four-dimensional (4D) intracardiac echocardiography (ICE) image-guided systems and methods, and more particularly to a system and method of integrating tracking elements into ultrasound catheters for tracking of the ultrasound catheters as part of a 4D ICE image-guided system.

Conventional catheter based techniques used in interventional procedures typically involve inserting a probe, such as an imaging catheter, into a vein, such as the femoral vein. Prior to performing catheter based interventional procedures, where an imaging catheter is used for either monitoring or treatment, precise guidance of the imaging catheter from the point of entry, through the vasculature of the patient to the desirable anatomical location is progressively becoming more important. Current techniques typically employ surgical navigation systems and fluoroscopy to monitor and guide the imaging catheter within the vasculature. Surgical navigation systems can track the position and orientation of a medical device or instrument and convey this location to a user. The position and orientation information can be conveyed by virtually superimposing a graphic representation of the medical device or instrument onto a patient image. Accordingly, the user receives visual feedback to help navigate or guide the medical device or instrument to a target site.

Conventional electromagnetic surgical navigation tracking systems generally include a tracking element disposed within a medical device or instrument such that the tracking element does not interfere with the performance of the medical procedure. The tracking element may comprise a magnetic field generator or a magnetic field sensor, and may comprise one or more coils defining a variety of different coil configurations. As medical personnel are frequently more concerned with the position and orientation of the medical device or instrument, it is often necessary to estimate the distance between a tracking element disposed near the proximal end of an instrument and the instrument's distal tip. One problem associated with tracking a catheter-based medical instrument is that catheters are flexible which may cause the distance between the proximal end and the distal end to vary. Another problem associated with tracking an ultrasound catheter is related to interference from the ultrasound motor that can render any tracking system position and orientation estimates less precise. A further problem associated with tracking a catheter is its small size and the ability to integrate and position the tracking elements within the catheter in order to track the position and orientation of the catheter with six degrees of freedom including positions along each of three primary axes x, y and z, and orientations or degree of rotations about each of the three primary axes roll, pitch and yaw.

4D ICE image-guided systems provide an essential tool for real-time intracardiac imaging. A 4D ICE imaging catheter includes a motor for rotating a 2D ultrasound transducer. Commonly used electromagnetic surgical navigation tracking systems generally operate from a frequency of DC up to 30 kHz. Due to the compact catheter packaging, the electromagnetic tracking elements need to be placed near the motor. This increases the risk of electromagnetic interference between the tracking elements and the motor. Thus, it is critical to address a strategy for improving electromagnetic tracking system compatibility with the 4D ICE imaging catheter motor.

Therefore, there is a need for a system and method for tracking the position and orientation of a catheter utilized during 4D ICE procedures with six degrees of freedom while simultaneously reducing the impact of noise generated from the 4D ICE imaging catheter motor.

### BRIEF DESCRIPTION OF THE INVENTION

In accordance with an aspect of the disclosure, an ultrasound catheter comprising a flexible catheter housing having a distal end; a transducer array disposed at least partially within the catheter housing; a motor coupled with the transducer array, said motor being configured to rotate the transducer array in order to image a three-dimensional (3D) volume; a first tracking element disposed within the catheter housing; and a second tracking element disposed around the first tracking element; wherein the first tracking element and the second tracking element are adapted to provide a position and orientation of the distal end of the catheter housing.

In accordance with an aspect of the disclosure, a surgical navigation system comprising an ultrasound catheter comprising a flexible catheter housing defining a distal end; a transducer array disposed at least partially within the catheter housing; and a motor coupled to transducer array, the motor being configured to rotate the transducer array in order to image a three-dimensional (3D) volume; a first tracking element disposed within the catheter housing; a second tracking element disposed within the catheter housing, the second tracking element being positioned and/or oriented differently from the first tracking element; and a tracking system coupled to the first tracking element and the second tracking element for tracking the position and orientation of the first tracking element, the second tracking element, and the catheter housing.

In accordance with an aspect of the disclosure, a method of tracking an ultrasound catheter comprising positioning a first tracking element within an ultrasound catheter; positioning a second tracking element within the ultrasound catheter; simultaneously tracking and calculating the position and orientation of the first tracking element and the second tracking element; calculating the position and orientation of the ultrasound catheter using the position and orientation data from the first tracking element and the second tracking element; and visualizing the position and orientation of the ultrasound catheter on a display of a preoperative or intraoperative image of a region of interest; wherein the second tracking element is positioned and/or oriented differently from the first tracking element within the ultrasound catheter.

Various other features, aspects, and advantages will be made apparent to those skilled in the art from the accompanying drawings and detailed description thereof.

### BRIEF DESCRIPTION OF THE DRAWINGS

FIG. 1 is a schematic representation of an exemplary embodiment of an imaging and navigation system;

FIG. 2 is a partially cutaway schematic representation of an exemplary embodiment of a trackable catheter;

FIG. 3 is a partially cutaway schematic representation of an exemplary embodiment of a trackable catheter;

FIG. 4 is a partially cutaway schematic representation of an exemplary embodiment of a trackable catheter;

FIG. 5 is a partially cutaway schematic representation of an exemplary embodiment of a trackable catheter;

FIG. 6 is a partially cutaway schematic representation of an exemplary embodiment of a trackable catheter;

FIG. 7 is a partially cutaway schematic representation of an exemplary embodiment of a trackable catheter; and

FIG. 8 is a flow diagram of an exemplary embodiment of a method of tracking an ultrasound catheter with six degrees of freedom.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the disclosure.

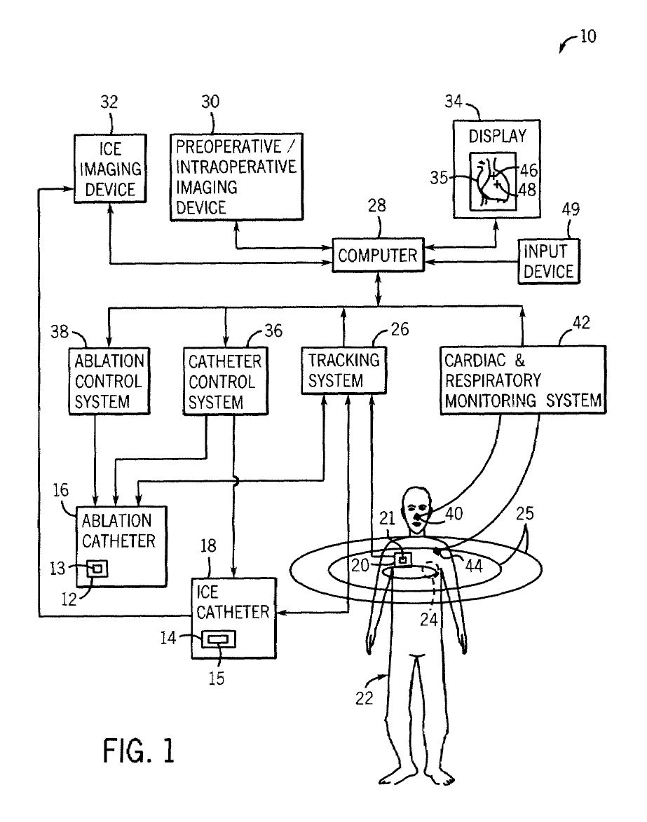

Referring to the drawings, FIG. 1 illustrates a schematic representation of an exemplary embodiment of an imaging and navigation system 10. The imaging and navigation system 10 will hereinafter be described as an imaging and navigation system adapted for treating atrial fibrillation using an ablation procedure. The imaging and navigation system 10 will also hereinafter be described as implementing intracardiac echocardiography (ICE) to facilitate the performance of the ablation procedure. It should, however, be appreciated that the imaging and navigation system 10 may also be implemented to treat other medical conditions and to perform other procedures, and that the imaging and navigation system 10 may implement alternate ultrasonic technologies in place of ICE.

The navigation portion of the imaging and navigation system 10 includes a tracking system 26 that is operatively connected to a plurality of tracking elements 12, 14 and 20. The tracking element 12 is adapted for attachment to an ablation catheter 16, and the tracking element 14 is adapted for attachment to an ICE catheter 18. For purposes of this disclosure, a catheter is defined to include any flexible medical delivery system such as, for example, any thin flexible tube for insertion into a body cavity, duct, or vessel. The tracking element 20 may be attached to an internal organ (e.g., the heart 24) or to the external body of the patient 22 in a conventional manner. The tracking element 20 secured to the patient's heart 24 may be referred to as a “dynamic reference” because it is adapted to move along with the heart 24. An exemplary method of attaching the tracking element 20 to the patient's heart 24 is through a minimally invasive procedure using a dynamic reference catheter (not shown).

In an exemplary embodiment, the tracking element 20 may comprise a field generator 21, the tracking element 12 may comprise a field sensor 13, and the tracking element 14 may comprise a field sensor 15. It should, however, be appreciated that according to alternate embodiments the tracking element 20 may comprise a field sensor and the tracking elements 12, 14 may comprise field generators. The field generator 21 generates a magnetic field 25 in an area that includes the target site (e.g., the patient's heart 24). The field sensors 13, 15 are adapted to measure the magnetic field 25, and to transmit the magnetic field measurements to the tracking system 26. The tracking system 26 and/or the computer 28 implement the magnetic field measurements to calculate the position and orientation of the tracking elements 12, 14.

After calculating the position and orientation of the tracking elements 12, 14, the position and orientation of the ablation catheter 16 and the ICE catheter 18 respectively attached thereto can also be calculated in a known manner. In an exemplary embodiment, the physical distance between the tracking elements 12, 14 and the distal end portions of the respective catheters 16, 18 are calculated and a translation/rotation matrix is applied as a conversion factor to the raw data from the tracking system 26. In (his manner, an operator can track with a high degree of precision both the position and orientation of the distal end portions of the catheters 16, 18.

The computer 28 registers the position and orientation data to an image obtained from a preoperative/intraoperative imaging device 30 and/or to an image obtained from an ICE imaging device 32. The preoperative/intraoperative imaging system 30 may, for example, include a computed tomography (CT) imaging device, a magnetic resonance (MR) imaging device, a positron emission tomography (PET) imaging device, an ultrasound imaging device, an X-ray imaging device, or any other known imaging device, as well as any combinations thereof. The preoperative/intraoperative imaging device 30 may provide two-dimensional (2D), three-dimensional (3D) or four-dimensional (4D) images. For purposes of this disclosure, 4D refers to the three primary dimensions (i.e., as measured along x, y and z axes) and the fourth dimension, which is time. Therefore, for purposes of this disclosure, 4D is synonymous with generally real-time 3D. Also for purposes of this disclosure, a generally real-time image includes a maximum image delay of approximately one second. The ICE imaging device 32 is configured to obtain imaging data from the ICE catheter 18 and produce 2D, 3D or 4D images as will be described in detail hereinafter.

The catheter position and orientation data can be visualized on the display 34. In an exemplary embodiment, graphic representations corresponding to the ablation catheter 16 and the ICE catheter 18 may be virtually superimposed on a patient image 35. In the embodiment of FIG. 1, the graphic representations corresponding to the catheters 16, 18 include the cross-hairs 46, 48 respectively representing the distal end portions of the ablation catheter 16 and the ICE catheter 18, however other embodiments may include a more complete rendering showing the catheters 16, 18 in detail. In a non-limiting manner, the patient image 35 may include a CT image, a MR image, a PET image, an ultrasound image or an X-ray image, or any combination thereof from the preoperative/intraoperative imaging device 30. The patient image 35 may also include a real time 3D image from the ICE imaging device 32, or a fused image comprising a plurality of images from the preoperative/intraoperative imaging device 30 and/or the ICE imaging device 32 that have been combined in a known manner.

The input device 49 may include any known apparatus or system such as a keyboard, mouse, touch screen, joystick, etc., and is generally adapted to allow a user to manually input data into the system 10. Although shown in FIG. I as a separate component, the input device 49 may alternatively be incorporated into one of the other system 10 components such as the computer 28 or the display 34. As an example, the input device 49 may include a touch screen device integrated into the design of the display 34 and adapted to facilitate surgical planning. In an exemplary embodiment, the exemplary touch screen input device 49 could be implemented to highlight or otherwise identify specific regions of interest on a patient image obtained from one of the imaging devices 30, 32. According to another embodiment, the exemplary touch screen input device 49 could be implemented to assign a priority sequence to a plurality of regions of interest.

A catheter control system 36 is operatively connected to both the ablation catheter 16 and the ICE catheter 18. The catheter control system 36 is adapted to translate and steer the catheters 16, 18 through the patient 22 to a predefined destination at or near the patient's heart 24. The catheter control system 36 may be configured to translate and steer the catheters 16, 18 in response to manual operator inputs, or may be configured to automatically direct the catheters 16, 18 to a selectable target site. The catheter control system 36 may also be operatively connected to and configured to control a dynamic reference catheter (not shown) adapted to facilitate the attachment of the tracking element 20 to the patient's heart 24.

An ablation control system 38 controls the energy transfer to the ablation catheter 16. Accordingly, when an operator determines that the distal end of the ablation catheter 16 is in sufficiently close proximity to a targeted cardiac region, the ablation control system 38 can be implemented to transmit a selectable amount of energy. The transmission of energy in this manner kills or otherwise renders inactive the targeted region in order to break electrical pathways causing atrial fibrillation. In a non-limiting manner, the ablation control system 38 may implement radio frequency (RF), cryogenic, ultrasound, or laser technologies.

One or more respiratory sensors 40 can be positioned near the patient's mouth and/or nose in order to monitor respiration, and one or more cardiac sensors 44 can be positioned near the patient's heart 24 to monitor cardiac activity. The respiratory sensors 40 and the cardiac sensors 44 are operatively associated with and adapted to transmit sensor data to a monitoring system 42. Any sensor data collected by the monitoring system 42 is transferable to the computer 28 such that the computer 28 may be implemented to synchronize the operation of the tracking system 26, the imaging device 30, and/or the imaging device 32 with the patient's cardiac and respiratory activity. According to one example, the computer 28 may implement data from the monitoring system 42 to acquire images during predefined portions of a patient's cardiac or respiratory cycle. According to another example, the computer 28 may implement data from the monitoring system 42 to sequence a series of 2D images or slices in a manner that corresponds with a patient's cardiac or respiratory cycle in order to provide a generally real time rendering of a dynamic object such as the patient's heart 24.

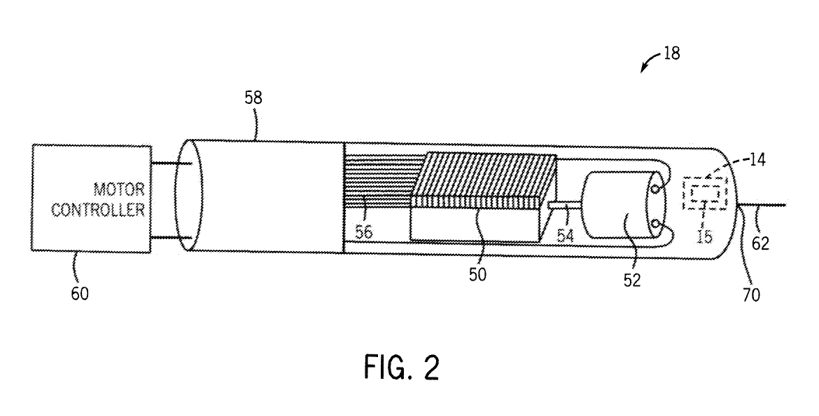

FIG. 2 is a more detailed illustration of the ICE catheter 18 is shown in accordance with an exemplary embodiment. It should be appreciated that the ICE catheter 18 is described for illustrative purposes, and that any catheter system adapted to retain an ultrasonic imaging device may alternatively be implemented in place of the ICE catheter 18.

The ICE catheter 18 comprises a transducer array 50, a motor 52, which may be internal or external to the space-critical environment, a drive shaft 54 or other mechanical connections between motor 52 and the transducer array 50, and an interconnect 56. The ICE catheter 18 further includes a catheter housing 58 enclosing the transducer array 50, motor 52, interconnect 56 and drive shaft 54. In the depicted embodiment, the transducer array 50 is mounted on drive shaft 54 and the transducer array 50 is rotatable with the drive shaft 54. The rotational motion of the transducer array 50 is controlled by motor controller 60 and motor 52. Interconnect 56 refers to, for example, cables, wires and other connections coupling the transducer array 50 with the ICE imaging device 32 shown in FIG. I for use in receiving and/or transmitting signals. In an exemplary embodiment, interconnect 56 is configured to reduce its respective torque load on the transducer array 50 and motor 52. The catheter housing 58 is of a material, size and shape adaptable for internal imaging applications and insertion into regions of interest. According to the embodiment depicted in FIG. 2, the catheter housing 58 is generally cylindrical defining a longitudinal axis 62.

The catheter housing 58, or at least the portion that intersects the ultrasound imaging volume, is acoustically transparent, e.g. low attenuation and scattering, acoustic impedance near that of blood and tissue (Z˜1.5M Rayl). The space between the transducer and the housing can be filled with an acoustic coupling fluid (not shown), e.g., water, with acoustic impedance and sound velocity near those of blood and tissue (Z˜1.5 M Rayl, V˜1540 m/sec).

In an exemplary embodiment, the transducer array 50 is a 64-element one-dimensional array having 0.110 mm azimuth pitch, 2.5 mm elevation and 6.5 MHz center frequency. The elements of the transducer array 50 are electronically phased in order to acquire a sector image parallel to the longitudinal axis 62 of the catheter housing 58. The transducer array 50 is mechanically rotated about the longitudinal axis 62 to image a 3D volume. The transducer array 50 captures a plurality of 2D images as it is being rotated. The plurality of 2D images are transmitted to the ICE imaging device 32 (shown in FIG. 1) which is configured to sequentially assemble the 2D images in order to produce a 3D image.

The rate at which the transducer array 50 is rotated about the longitudinal axis 62 can be regulated by the motor controller 60. The transducer array 50 can be rotated relatively slowly to produce a 3D image, or relatively quickly to produce a generally real-time 3D image (i.e., a 4D image). The motor controller 60 is also operable to vary the direction of rotation to produce an oscillatory transducer array motion. In this manner, the range of motion and imaged volume are restricted such that the transducer array 50 can focus on imaging a specific region and can update the 3D image of that region more frequently, thereby providing a real-time 3D, or 4D, image.

Referring to FIGS. 1 and 2, the ICE catheter 18 includes an integrally attached tracking element 14 disposed within the catheter housing 58. The integrally attached tracking element 14 is adapted to work in combination with the tracking element 20 and the tracking system 26 to estimate the position and orientation of the ICE catheter 18. While the tracking element 14 is depicted as comprising the field sensor 15 in accordance with one embodiment, it should be appreciated that the tracking element 14 may alternatively comprise a field generator (not shown) similar to the field generator 21.

The field sensor 15 and field generator may comprise at least one electromagnetic microsensor adapted to track the position and orientation of the ICE catheter 18 with six degrees of freedom. For purposes of this disclosure, the six degrees of freedom refer to the position along each of three primary x, y and z axes, and the orientation or degree of rotation about each of three primary pitch, roll and yaw axes.

In an exemplary embodiment, the at least one electromagnetic microsensor may be defined by various architectures, including various coil architectures and other electromagnetic sensor architectures. In the case of various coil architectures, the at least one electromagnetic microsensor may comprise single coils, a pair of single coils, industry-standard-coil-architecture (ISCA) type coils, a pair of ISCA type coils, multiple coils, or-an array of coils.

ISCA type coils are defined as three approximately collocated, approximately orthogonal, and approximately dipole coils. Therefore, ISCA electromagnetic transmitter and receiver coils would include three approximately collocated, approximately orthogonal, and approximately dipole coils for the transmitter assembly and three approximately collocated, approximately orthogonal, and approximately dipole coils for the receiver assembly. In other words, an ISCA configuration for the electromagnetic transmitter and receiver assemblies would include a three-axis dipole coil transmitter and a three-axis dipole coil receiver. In the ISCA configuration, the transmitter coils and the receiver coils are configured such that the three coils (i.e., coil trios) exhibit the same effective area, are oriented orthogonally to one another, and are centered at the same point.

In the case of other electromagnetic sensor architectures, the at least one electromagnetic microsensor may comprise flux gate magnetometer sensors, squid magnetometer sensors, Hall-effect sensors, anisotropic magneto-resistance (AMR) sensors, giant magneto-resistance (GMR) sensors, or extraordinary magneto-resistance (EMR) sensors.

In an exemplary embodiment, the field sensor 15 may be a wireless field sensor or a wired field sensor. In an exemplary embodiment, the field generator may be a wireless field generator or a wired field generator.

Returning to FIG. 2, the tracking element 14 is positioned immediately adjacent to a distal end 70 of the catheter housing 58. For purposes of this disclosure, the term “immediately adjacent” refers to the depicted arrangement wherein there are no other components disposed between the tracking element 14 and the distal end 70.

As previously indicated, the imaging and navigation system 10 may estimate the physical distance between the tracking element 14 and the distal end 70 of the catheter housing 58, and implement this estimate as a conversion factor to the raw data from the tracking system 26 in order to track the position and orientation of the distal end 70 of the ICE catheter 18. The process of estimating the physical distance between the tracking element 14 and the distal end 70 of the catheter housing 58 is complicated by the fact that the catheter housing 58 is flexible thereby potentially causing the estimated distance to vary. By positioning the tracking element 14 near the distal end 70 of the catheter housing 58, the distance between the tracking element 14 and the distal end 70 remains generally constant regardless of catheter housing 58 flex. Accordingly, the precision with which the tracking system 26 estimates the position and orientation of the distal end 70 of the ICE catheter 18 is improved.

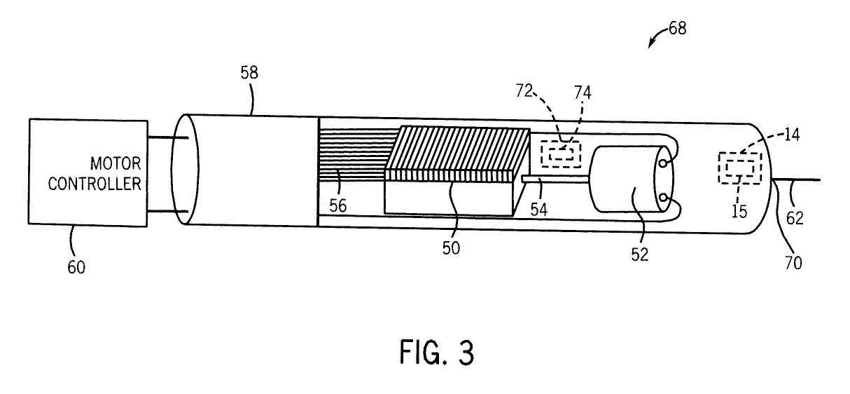

FIG. 3 illustrates an ICE catheter 68 in accordance with an exemplary embodiment. Common reference numbers are implemented to identify ICE catheter 68 elements that are generally identical to those of the ICE catheter 18 shown in FIG. 2. It should be appreciated that the ICE catheter 68 is described for illustrative purposes, and that any catheter system adapted to retain an ultrasonic imaging device may alternatively be implemented in place of the ICE catheter 68.

The ICE catheter 68 includes all the components and features previously described with respect to the ICE catheter 18 shown in FIG. 2, and additionally includes a tracking element 72. The tracking element 72 will hereinafter be described as comprising a field sensor 74 in accordance with one embodiment, however it should be appreciated that the tracking element 72 may alternatively comprise a field generator (not shown) similar to the field generator 21 shown in FIG. 1.

The field sensor 74 and field generator may comprise at least one electromagnetic microsensor adapted to track the position and orientation of the ICE catheter 68 with six degrees of freedom (x, y, z, pitch, roll, yaw).

In an exemplary embodiment, the at least one electromagnetic microsensor may be defined by various architectures, including various coil architectures and other electromagnetic sensor architectures. In the case of various coil architectures, the at least one electromagnetic microsensor may comprise single coils, a pair of single coils, ISCA type coils, a pair of ISCA type coils, multiple coils, or an array of coils. In the case of other electromagnetic sensor architectures, the at least one electromagnetic microsensor may comprise flux gate magnetometer sensors, squid magnetometer sensors, Hall-effect sensors, AMR sensors, GMR sensors, or EMR sensors.

In an exemplary embodiment, the field sensor 74 may be a wireless field sensor or a wired field sensor. In an exemplary embodiment, the field generator may be a wireless field generator or a wired field generator.

According to the depicted embodiment shown in FIG. 3, the tracking element 72 is disposed within the catheter housing 58, and is positioned behind and in close proximity to the motor 52. According to another exemplary embodiment that is not shown, the tracking element 72 may be positioned in front of and in close proximity to the motor 52. By positioning the tracking element 72 in close proximity to the motor 52, the tracking element 72 may be implemented to track and record the noise signal attributable to motor 52 interference. By tracking and recording the noise signal attributable to motor 52 interference, the noise signal can be removed or otherwise accounted for in a known manner such that tracking system precision is improved. In an exemplary embodiment, the tracking system 26 is configured to receive the recorded noise signal from the tracking element 72, and to compensate for the noise signal in a manner adapted to provide position and orientation estimates that are not predicated on noise signal data.

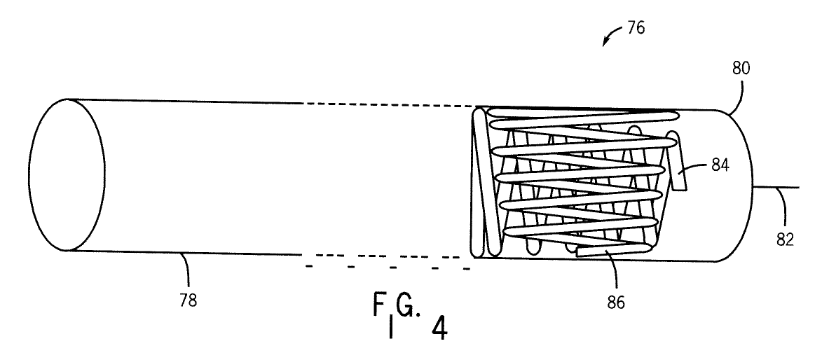

FIG. 4 illustrates a schematic representation of an exemplary embodiment of a trackable catheter 76. The catheter 76 comprises a catheter housing 78 having a distal end 80. The catheter housing 78 is of a material, size and shape adaptable for internal medical applications and insertion into regions of interest. According to the embodiment depicted in FIG. 4, the catheter housing 78 is generally cylindrical defining a longitudinal axis 82.

The catheter 76 includes a first tracking coil 84 and a second tracking coil 86 disposed within the catheter housing 78. The first tracking coil 84 is a single coil integrated lengthwise in the catheter housing 78 along the longitudinal axis 82. The second tracking coil 86 is a single coil wound around the first tracking coil 84 and oriented perpendicular to the longitudinal axis 82. The first tracking coil 84 is adapted to work in combination with the second tracking coil 86 and the tracking system 26 to estimate the position and orientation of the catheter 76 with six degrees of freedom (x, y, z, pitch, roll, yaw). In an exemplary embodiment, the first tracking coil 84 may be an electromagnetic single coil transmitter and the second tracking coil 86 may be an electromagnetic single coil receiver. In an exemplary embodiment, the first tracking coil 84 may be an electromagnetic single coil receiver and the second tracking coil 86 may be an electromagnetic single coil transmitter. In an exemplary embodiment, the first tracking coil 84 may be an electromagnetic single coil transmitter and the second tracking coil 86 may be an electromagnetic single coil transmitter. In an exemplary embodiment, the first tracking coil 84 may be an electromagnetic single coil receiver and the second tracking coil 86 may be an electromagnetic single coil receiver.

The first tracking coil 84 and the second tracking coil 86 may be positioned anywhere along the length of the catheter housing 78. As previously indicated, the imaging and navigation system 10 may estimate the physical distance between the first and second tracking coils 84, 86 and the distal end 80 of the catheter housing 78, and implement this estimate as a conversion factor to the raw data from the tracking system 26 in order to track the position and orientation of the distal end 80 of the catheter 76.

In an exemplary embodiment, the first and second tracking coils 84, 86 may be precisely manufactured or precisely characterized during manufacture to obtain mathematical models of the first and second tracking coils 84, 86. From the magnetic field measurements and mathematical models of the first ands second tracking coils 84, 86, the position and orientation of the catheter 76 may be determined.

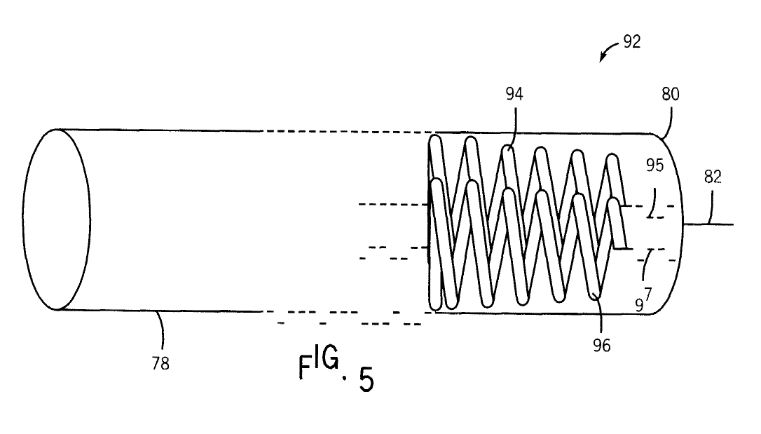

FIG. 5 illustrates a schematic representation of an exemplary embodiment of a trackable catheter 92. Common reference numbers are implemented to identify catheter elements that are generally identical to those of the catheter shown in FIG. 4. The catheter includes all the components and features previously described with respect to the catheter shown in FIG. 4. For example, the catheter 92 comprises a catheter housing 78 having a distal end 80 and a longitudinal axis 82.

The catheter 92 includes a first tracking coil 94 and a second tracking coil 96 disposed within the catheter housing 78. The first tracking coil 94 is a single coil integrated lengthwise in the catheter housing 78 along a first longitudinal axis 95 that is parallel to longitudinal axis 82 of the catheter 92. The second tracking coil 96 is a single coil wound around the first tracking coil 94 and also integrated lengthwise in the catheter housing 78 along a second longitudinal axis 97 that is parallel to longitudinal axis 95 and longitudinal axis 82. The first and second tracking coils 94, 96 are aligned in the same direction but separated radially (i.e., side-by-side) from each other. The first tracking coil 94 is adapted to work in combination with the second tracking coil 96 and the tracking system 26 to estimate the position and orientation of the catheter 92 with six degrees of freedom (x, y, z, pitch, roll, yaw). In an exemplary embodiment, the first tracking coil 94 may be an electromagnetic single coil transmitter and the second tracking coil 96 may be an electromagnetic single coil receiver. In an exemplary embodiment, the first tracking coil 94 may be an electromagnetic single coil receiver and the second tracking coil 96 may be an electromagnetic single coil transmitter. In an exemplary embodiment, the first tracking coil 94 may be an electromagnetic single coil transmitter and the second tracking coil 96 may be an electromagnetic single coil transmitter. In an exemplary embodiment, the first tracking coil 94 may be an electromagnetic single coil receiver and the second tracking coil 96 may be an electromagnetic single coil receiver.

The first tracking coil 94 and the second tracking coil 96 may be positioned anywhere along the length of the catheter housing 78. As previously indicated, the imaging and navigation system 10 may estimate the physical distance between the first and second tracking coils 94, 96 and the distal end 80 of the catheter housing 78, and implement this estimate as a conversion factor to the raw data from the tracking system 26 in order to track the position and orientation of the distal end 80 of the catheter 92.

In an exemplary embodiment, the first and second tracking coils 94, 96 may be precisely manufactured or precisely characterized during manufacture to obtain mathematical models of the first and second tracking coils 94, 96. From the magnetic field measurements and mathematical models of the first ands second tracking coils 94, 96, the position and orientation of the catheter 92 may be determined.

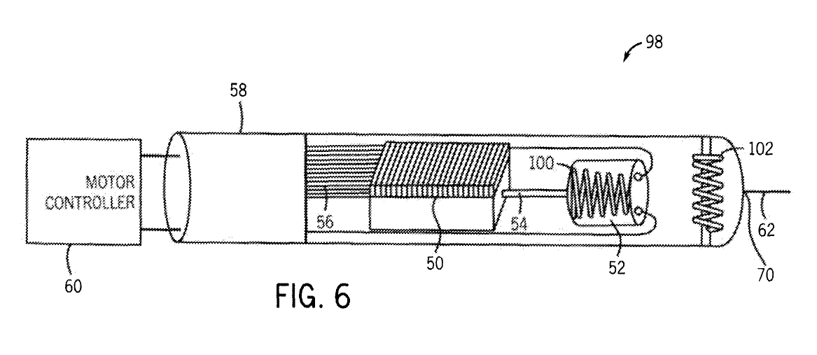

FIG. 6 illustrates an ICE catheter 98 in accordance with an exemplary embodiment. Common reference numbers are implemented to identify ICE catheter 98 elements that are generally identical to those of the ICE catheter shown in FIGS. 2 and 3. It should be appreciated that the ICE catheter 98 is described for illustrative purposes, and that any catheter system adapted to retain an ultrasonic imaging device may alternatively be implemented in place of the ICE catheter 98.

The ICE catheter 98 includes all the components and features previously described with respect to the ICE catheter shown in FIGS. 2 and 3. The ICE catheter 98 further includes a first tracking coil 100 wound around the motor 52 to maximize the coil effective area and conserving spatial occupation of the catheter 98, and a second tracking coil 102 disposed within the catheter housing 58 and positioned adjacent to the distal end 70 of the catheter housing 58. In an exemplary embodiment, the first tracking coil 100 is a single coil oriented around and in line with the longitudinal axis 62. In an exemplary embodiment, the second tracking coil 102 is a single coil and oriented perpendicular to the longitudinal axis 62. In an exemplary embodiment, the second tracking coil 102 is positioned in front of and in close proximity to the motor 52.

By positioning the second tracking coil 102 in close proximity to the motor 52, the second tracking coil 102 may be implemented to track and record the noise signal attributable to motor 52 interference. By tracking and recording the noise signal attributable to motor 52 interference, the noise signal can be removed or otherwise accounted for in a known manner such that tracking system precision is improved. In an exemplary embodiment, the tracking system 26 is configured to receive the recorded noise signal from the second tracking coil 102, and to compensate for the noise signal in a manner adapted to provide position and orientation estimates that are not predicated on noise signal data.

The first tracking coil 100 is adapted to work in combination with the second tracking coil 102 and the tracking system 26 to estimate the position and orientation of the ICE catheter 98 with six degrees of freedom (x, y, z, pitch, roll, yaw). In an exemplary embodiment, the first tracking coil 100 may be an electromagnetic single coil transmitter and the second tracking coil 102 may be an electromagnetic single coil receiver for the tracking system 26. In an exemplary embodiment, the first tracking coil 100 may be an electromagnetic single coil receiver and the second tracking coil 102 may be an electromagnetic single coil transmitter for the tracking system 26.

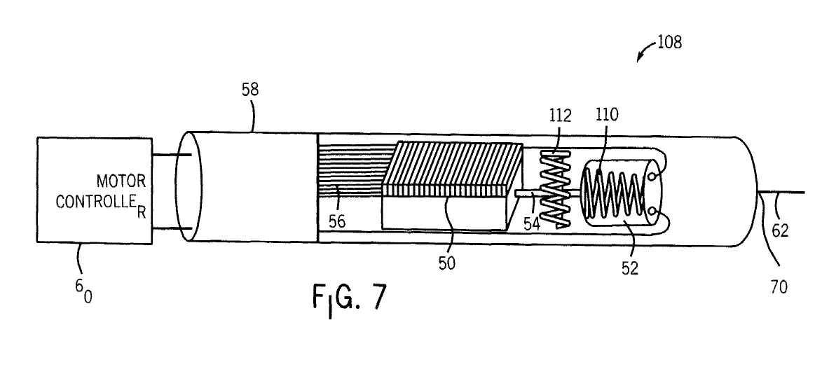

FIG. 7 illustrates an ICE catheter 108 in accordance with an exemplary embodiment. Common reference numbers are implemented to identify ICE catheter 108 elements that are generally identical to those of the ICE catheter shown in FIGS. 2 and 3. It should be appreciated that the ICE catheter 108 is described for illustrative purposes, and that any catheter system adapted to retain an ultrasonic imaging device may alternatively be implemented in place of the ICE catheter 108.

The ICE catheter 108 includes all the components and features previously described with respect to the ICE catheter shown in FIGS. 2 and 3. The ICE catheter 108 further includes a first tracking coil 110 wound around the motor 52 to maximize the coil effective area and conserving spatial occupation of the catheter 108, and a second tracking coil 112 disposed within the catheter housing 58 and positioned behind and in close proximity to the motor 52. In an exemplary embodiment, the first tracking coil I 10 is a single coil oriented around and in line with the longitudinal axis 62. In an exemplary embodiment, the second tracking coil 112 is a single coil and oriented perpendicular to the longitudinal axis 62.

By positioning the second tracking coil 112 in close proximity to the motor 52, the second tracking coil 112 may be implemented to track and record the noise signal attributable to motor 52 interference. By tracking and recording the noise signal attributable to motor 52 interference, the noise signal can be removed or otherwise accounted for in a known manner such that tracking system precision is improved. In an exemplary embodiment, the tracking system 26 is configured to receive the recorded noise signal from the second tracking coil 112, and to compensate for the noise signal in a manner adapted to provide position and orientation estimates that are not predicated on noise signal data.

The first tracking coil 110 is adapted to work in combination with the second tracking coil 112 and the tracking system 26 to estimate the position and orientation of the ICE catheter 108 with six degrees of freedom (x, y, z, pitch, roll, yaw). In an exemplary embodiment, the first tracking coil 110 may be an electromagnetic single coil transmitter and the second tracking coil 112 may be an electromagnetic single coil receiver for the tracking system 26. In an exemplary embodiment, the first tracking coil 110 may be an electromagnetic single coil receiver and the second tracking coil 112 may be an electromagnetic single coil transmitter for the tracking system 26.

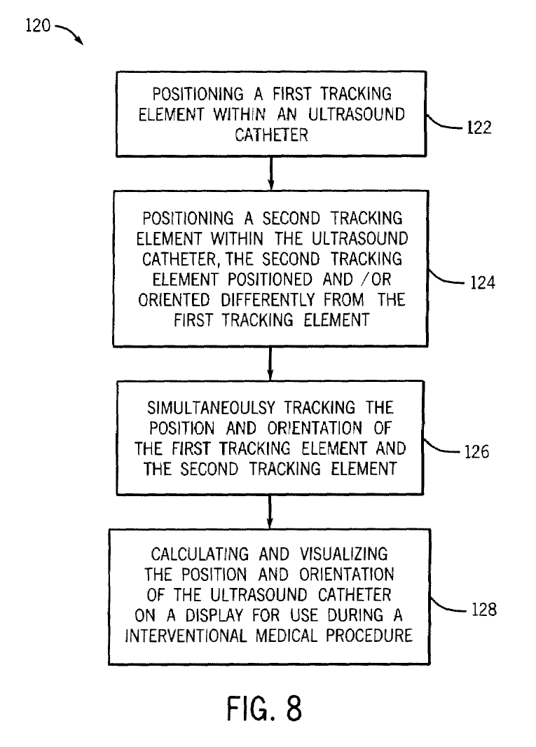

FIG. 8 is a flow diagram of an exemplary embodiment of a method 120 of tracking an ultrasound catheter with six degrees of freedom (x, y, z, pitch, roll, yaw). The method 120 includes positioning a first tracking element within an ultrasound catheter 122. The method 120 further includes positioning a second tracking element within the ultrasound catheter 124. The second tracking element is positioned and/or oriented differently within the ultrasound catheter from the first tracking element. A next step is simultaneously tracking and calculating the position and orientation of each of the first tracking element and the second tracking element with a tracking system 126. The final step is calculating and visualizing the position and orientation of the ultrasound catheter on a display of a preoperative or intraoperative image of a region of interest for use during an interventional medical procedure 128. The position and orientation of the ultrasound catheter is calculated using the position and orientation data from the first tracking element and the second tracking element.

In an exemplary embodiment, at least one of the first or second tracking elements may be positioned on or in close proximity with a motor of the ultrasound catheter, such that interference from the motor may be removed or accounted for (limited) in calculating the position and orientation of the ultrasound catheter.

Several embodiments are described above with reference to drawings. These drawings illustrate certain details of exemplary embodiments that implement the apparatus, assemblies, systems, and methods of this disclosure. However, the drawings should not be construed as imposing any limitations associated with features shown in the drawings.

The exemplary embodiments described herein provide specific, feasible apparatus, systems, and methods of integrating electromagnetically trackable microsensors into catheters that do not currently exist. By integrating microsensors into catheters in a robust and clinically effective way, minimally invasive surgical techniques and interventional procedures, can utilize electromagnetic tracking technology to provide more efficient treatments, less radiation dose, and faster procedures.

The exemplary embodiments of catheters described herein may be used as part of a surgical navigation system employing electromagnetic tracking technology that may be used in an interventional surgical suite. The surgical navigation system may be integrated into a fixed imaging system, a portable imaging system, or a stand-alone electromagnetic navigation tracking system.

The foregoing description of exemplary embodiments has been presented for purposes of illustration and description. It is not intended to be exhaustive or to limit the disclosure to the precise form disclosed, and modifications and variations are possible in light of the above teachings or may be acquired from practice of the disclosure. The embodiments were chosen and described in order to explain the principles of the disclosure and its practical application to enable one skilled in the art to utilize the invention in various embodiments and with various modifications as are suited to the particular use contemplated.

While the disclosure has been described with reference to various embodiments, those skilled in the art will appreciate that certain substitutions, alterations and omissions may be made to the embodiments without departing from the spirit of the disclosure. Accordingly, the foregoing description is meant to be exemplary only, and should not limit the scope of the disclosure as set forth in the following claims.

What is claimed is:

1. An ultrasound catheter comprising:
a flexible catheter housing having a distal end;
a transducer array disposed at least partially within the catheter housing;
a motor coupled with the transducer array, said motor being configured to rotate the transducer array in order to image a three-dimensional (3D) volume;
a first tracking element disposed within the catheter housing; and
a second tracking element disposed around the first tracking element;
wherein the first tracking element and the second tracking element are adapted to provide a position and orientation of the distal end of the catheter housing.

2. The ultrasound catheter of claim 1, wherein the first tracking element is a single coil.

3. The ultrasound catheter of claim 2, wherein the second tracking element is a single coil wound around the single coil of the first tracking element.

4. The ultrasound catheter of claim 3, wherein the single coil of the first tracking element is oriented along a first longitudinal axis.

5. The ultrasound catheter of claim 4, wherein the single coil of the second tracking element is oriented along a second longitudinal axis.

6. The ultrasound catheter of claim 5, wherein the second longitudinal axis is parallel to the first longitudinal axis and parallel to a longitudinal axis of the catheter housing.

7. The ultrasound catheter of claim 3, wherein the single coil of the second tracking element is oriented perpendicular to the single coil of the first tracking element and perpendicular to a longitudinal axis of the catheter housing.

8. The ultrasound catheter of claim 1, wherein the first tracking element is a single coil wound around the motor.

9. The ultrasound catheter of claim 1, wherein the second tracking element is a single coil disposed within the catheter housing at the distal end of the catheter housing.

10. The ultrasound catheter of claim 1, wherein the second tracking element is a single coil disposed within the catheter housing between the transducer array and the motor.

11. The ultrasound catheter of claim 1, wherein the first tracking element and the second tracking element are configured to track the catheter housing with six degrees of freedom.

12. A surgical navigation system comprising:
an ultrasound catheter comprising:
a flexible catheter housing defining a distal end;
a transducer array disposed at least partially within the catheter housing; and
a motor coupled to transducer array, the motor being configured to rotate the transducer array in order to image a three-dimensional (3D) volume;
a first tracking element disposed within the catheter housing;
a second tracking element disposed within the catheter housing, the second tracking element being positioned and/or oriented differently from the first tracking element; and
a tracking system coupled to the first tracking element and the second tracking element for tracking the position and orientation of the first tracking element, the second tracking element, and the catheter housing.

13. The surgical navigation system of claim 12, wherein the ultrasound catheter is an ICE catheter.

14. The surgical navigation system of claim 12, wherein the first tracking element and the second tracking element each comprise single coils.

15. The surgical navigation system of claim 12, wherein the first tracking element and the second tracking element are configured to track the catheter housing with six degrees of freedom.

16. The surgical navigation system of claim 14, wherein the second tracking element is wound around the first tracking element.

17. The surgical navigation system of claim 12, wherein the first tracking element is oriented along a first longitudinal axis.

18. The surgical navigation system of claim 17, wherein the second tracking element is oriented along a second longitudinal axis.

19. The surgical navigation system of claim 18, wherein the second longitudinal axis is parallel to the first longitudinal axis and parallel to a longitudinal axis of the catheter housing.

20. The surgical navigation system of claim 12, wherein the second tracking element is oriented perpendicular to the first tracking element and perpendicular to a longitudinal axis of the catheter housing.

21. The surgical navigation system of claim 12, wherein the first tracking element is a single coil wound around the motor.

22. The surgical navigation system of claim 12, wherein the second tracking element is disposed within the catheter housing at the distal end of the catheter housing.

23. The surgical navigation system of claim 12, wherein the second tracking element is disposed within the catheter housing between the transducer array and the motor.

24. The surgical navigation system of claim 12, further comprising a display adapted to visualize the position and orientation of the ultrasound catheter.

25. A method of tracking an ultrasound catheter comprising:
positioning a first tracking element within an ultrasound catheter;
positioning a second tracking element within the ultrasound catheter;
simultaneously tracking and calculating the position and orientation of the first tracking element and the second tracking element;
calculating the position and orientation of the ultrasound catheter using the position and orientation data from the first tracking element and the second tracking element; and
visualizing the position and orientation of the ultrasound catheter on a display of a preoperative or intraoperative image of a region of interest;
wherein the second tracking element is positioned and/or oriented differently from the first tracking element within the ultrasound catheter.

26. The method of tracking an ultrasound catheter of claim 25, wherein at least one of the first or second tracking elements is positioned on or in close proximity with a motor of the ultrasound catheter, such that interference from the motor may be removed in calculating the position and orientation of the ultrasound catheter.
